(19) United States
(12) Patent Application Publication
(10) Pub. No.: US 2022/0111354 A1
(43) Pub. Date: Apr. 14, 2022

(54) CATALYST AND PROCESS OF PREPARING THE SAME

(71) Applicant: Jawaharlal Nehru Centre for Advanced Scientific Research, Jakkur Bangalore (IN)

(72) Inventors: Sebastian Chirambatte PETER, Jakkur Bangalore (IN); Soumyabrata ROY, Jakkur Bangalore (IN); Arjun CHEREVOTAN, Jakkur Bangalore (IN); Manoj Kaja SAI, Jakkur Bangalore (IN)

(21) Appl. No.: 17/298,378

(22) PCT Filed: Nov. 29, 2019
(86) PCT No.: PCT/IN2019/050873

§ 371 Date: May 28, 2021

(30) Foreign Application Priority Data
Nov. 29, 2018 (IN) 201841045187

(51) Int. Cl. B01J 23/00; B01J 23/44; B01J 23/825; B01J 37/04; B01J 37/08; B01J 37/00; B01J 37/34; B01J 37/03; B01J 37/02; C01B 32/40; B01J 35/10; C07C 29/154

(52) U.S. Cl. CPC B01J 23/002; C01P 2004/04; B01J 23/825; B01J 37/04; B01J 37/082; B01J 37/009; B01J 37/343; B01J 37/031; B01J 37/0207; B01J 37/0009; C01B 32/40; B01J 35/1014; C07C 29/154; C01P 2002/72; C01P 2004/03; C01P 2002/85; B01J 23/44

(57) ABSTRACT

A catalyst including at least one group 10 metal impregnated in a mixed metal oxide is provided. The mixed metal oxide is selected from the group consisting of the oxides of In, Cu. Zn, Zr, Al, and combinations thereof. The catalyst is effective in the conversion of CO₂ to value added products like methanol (CH₃OH) and carbon monoxide (CO). A convenient process for preparation of the catalyst is also provided.

### CROSS-REFERENCE TO RELATED APPLICATIONS

This application is a Section 371 of International Application No. PCT/IN2019/050873, filed Nov. 29, 2019, which was published in the English language on Jun. 4, 2020, under International Publication No. WO 2020/110151 A1, which claims priority under 35 U.S.C. § 119(b) to Indian Application No. 201841045187, filed Nov. 29, 2018, the disclosure of each of which is incorporated herein by reference in their entireties.

### TECHNICAL FIELD

The subject matter described herein, in general, relates to catalysts, and in particular relates to a catalyst for conversion of carbon dioxide (CO₂) to value-added products.

### BACKGROUND OF INVENTION

Production of energy and its utilization in modern society are mostly based on the combustion of carbonaceous fuels like coal, petroleum and natural gas. However, complete combustion of these fuels produces carbon dioxide (CO₂), which alters the earth's carbon cycle and greatly enhances the atmospheric concentration of CO₂. Global warming caused by a remarkable increase in CO₂ emission into the atmosphere is an essential and urgent problem to be solved. Several strategies are under assessment for the reduction of CO₂ emission. One of the strategies to reduce CO₂ emission is to shift the fuel base from coal to gas. However, in geographically big countries like India, Australia, and China, which have large natural fossil fuel deposits, this shift of energy base to imported gas or oil will rarely be a convenient and practical choice. Another strategy of reducing CO₂ is its capture and storage; which is associated with high cost and energy-intensive requirements.

Catalytic conversion of CO₂ to value-added products such as carbon monoxide, hydrogen, methanol, acetaldehyde, glycerol, dimethyl ether, ethanol, acetone, etc., is one of the most viable and attractive processes to mitigate the greenhouse effect caused by the rise of CO₂ concentration in the atmosphere. From among the value-added products that can be obtained from catalytic conversion of CO₂, methanol, MeOH, is a highly desirable product owing to its wide applications in portable devices, fuel cells, feedstock for the synthesis of olefins, as fuel, etc. Two different synthetic routes for methanol production from CO₂ and H₂ are known; a direct route where methanol is directly produced from CO₂ and hydrogen, and an indirect route where CO₂ is first converted into carbon monoxide (CO) via reverse water-gas shift (RWGS) reaction to produce syngas. The syngas is further transported to a reactor to convert into methanol (Anicic, B.; Energy 2014, 77, 279-289).

MeOH Production from CO₂

CO₂+3H₂↔CH₃OH+H₂O ΔH=−49.5 kJ/mol (Equation 1)

Reverse Water-Gas Shift reaction (RWGS)

CO₂+H₂↔CO+H₂O ΔH=42.1 kJ/mol (Equation 2)

These reactions are conventionally catalyzed by catalysts like Cu/ZnO/Al₂O₃ (in different compositional variations), often with promoters like CeO₂, ZrO₂, SiO₂, Cr₂O₃ or Ga₂O₃, where the CO₂ conversion feed is about 25%. (JP-A-H07-39755 and JP-A-H06-312138). However, a large amount of water formed as a by-product, from both methanol production and the RWGS side reaction, has an inhibitory effect on the active metal (catalyst) during the reaction leading to the deactivation of the catalyst. Besides, several side products such as higher alcohols and hydrocarbons are usually formed in this reaction. Therefore, methanol synthesis from CO₂ hydrogenation requires a more selective catalyst to avoid the formation of undesired by-products.

More recently a couple of interesting intermetallic compounds were reported: Ni₅Ga₃ (Studt et al, Nat. Chem., 2014, 6, 320-324) and Pd₂Ga (Fiordaliso et al., ACS Catal., 2015, 5, 5827-5836) with selectivity much higher than the state-of-the-art material Cu/ZnO₂/Al₂O₃. EP2680964 discloses a catalytic composition for methanol production, comprising an alloy of at least two different metals M and M′, where M is selected from Ni, Pd, Fe, and Ru, and M′ is selected from Ga, Zn, and Al. CA2126502 discloses a catalyst for the reduction of carbon dioxide which comprises at least one transition metal selected from the group consisting of Group 8 (e. g., Ni, Fe, Co, Ru, Rh) and Group VIA (e.g., Mo, W) in the periodic table on zinc oxide alone or a composite containing zinc oxide and at least one metal oxide of a metal selected from the group consisting of Group 3B (e.g., Al, Ga) and Group 4A (e.g., Ti, Zr) in the periodic table.

Although many efforts have been made to facilitate the progress of methanol synthesis from CO₂ hydrogenation through the use of various catalysts, there still exists a need to develop a cost-effective catalyst with higher conversion efficiency, improved selectivity, and stability.

### BRIEF SUMMARY OF THE INVENTION

In an aspect of the present invention, there is provided a catalyst comprising at least one group 10 metal impregnated in a mixed metal oxide selected from the group consisting of the oxides of In, Cu, Zn, Zr, Al, and combinations thereof.

In another aspect of the present disclosure, there is provided a process for preparing a catalyst comprising at least one group 10 metal impregnated in a mixed metal oxide selected from the group consisting of the oxides of In, Cu, Zn, Zr, Al, and combinations thereof, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture; d) processing the first reaction mixture to obtain a mixed metal oxide; e) contacting the mixed metal oxide and an aqueous solution of palladium nitrate to obtain a second solution; and f) processing the second solution to obtain the catalyst.

In yet another aspect of the present disclosure, there is provided use of the catalyst comprising at least one group 10 metal impregnated in a mixed metal oxide selected from the group consisting of the oxides of In, Cu, Zn, Zr, Al, and combinations thereof, for thermochemical CO₂ reduction.

In a further aspect of the present disclosure, there is provided a catalyst comprising at least one group 10 metal impregnated in a mixed metal oxide selected from the group consisting of the oxides of In, Cu, Zn, Zr, Al, and combinations thereof, for use in thermochemical CO₂ reduction.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

The patent or application file contains at least one drawing executed in color. Copies of this patent or patent application publication with color drawings will be provided by the Office upon request and payment of the necessary fee.

FIG. 1 illustrates characterization data of the catalyst (a) Powder X-ray Diffraction (PXRD) of PdO—In₂O₃/CuO/ZnO/ZrO₂/Al₂O₃ (catalyst) and comparison with bulk oxides, (b) PXRD of the catalyst after reduction, (c) Scanning Electron Microscope (SEM) before annealing, (d) SEM after annealing (e) SEM and Energy-Dispersive X-ray Spectroscopy (EDS), (f) High-Resolution Transmission Electron Microscopy (HRTEM), (g) Transmission Electron Microscopy (TEM), (h) temperature programmed reduction data (i) HRTEM showing d-spacing for CuO (110) plane and (j) Selected Area Electron Diffraction (SAED) pattern on a region of the catalyst, in accordance with an embodiment of the present subject matter.

FIG. 2 depicts calibration curves for (a) CO, (b) CO, (c) MeOH, (d) CO₂ and (e) N₂, in accordance with an embodiment of the present disclosure.

FIG. 3 depicts 72-hour steady state experimental data of (a) conversion and temperature vs. time at 1000 Gas Hourly Space Velocity (GHSV), (b) selectivity and temperature vs. time at 1000 GHSV, (c) conversion vs. time at 1000, 2400, and 3600 GHSV, (d) conversion and temperature vs. time at 3600 GHSV, (e) selectivity and temperature vs. time at 3600 GHSV and (f) conversion vs temperature at 1000, 2400, and 3600 GHSV, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight percentage of about 50-59% should be interpreted to include not only the explicitly recited limits of about 50% to about 59%, but also to include sub-ranges, such as 51%, 52.5%, 54%, 58.99% and so forth.

The term “at least one” is used to mean one or more and thus includes individual components as well as mixtures/combinations.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

Conventionally used catalysts for catalytic hydrogenation of CO₂ to MeOH are associated with a low CO₂ conversion efficiency and less selectivity for the desired products. This is because CO₂ conversion is a thermodynamic equilibrium limited reaction, where a large amount of water is generated as a by-product along with methanol, which leads to the de-activation of the catalyst. Certain other conventionally used catalysts get degraded in even with 1 ppm of sulfur compounds; and are associated with poor durability and high costs. Considering the factors of conversion efficiency, selectivity, stability, catalyst sensitivities to impurities, and cost, there exists a need to develop a catalyst that overcomes the drawbacks of the art. Accordingly, the present disclosure describes a catalyst comprising at least one group 10 metal impregnated in a combination of mixed metal oxides. The catalysts of the present disclosure allow for efficient thermo-catalytic conversion of CO₂ to MeOH and CO with higher selectivity, in an energy efficient manner.

In an embodiment of the present disclosure, there is provided a catalyst comprising at least one group 10 metal impregnated in a mixed metal oxide selected from the group consisting of the oxides of In, Cu, Zn, Zr, Al, and combinations thereof.

In an embodiment of the present disclosure, there is provided a catalyst as described herein, wherein the mixed metal oxide is a combination of In₂O₃, CuO, ZnO, ZrO₂, and Al₂O₃.

In an embodiment of the present disclosure, there is provided a catalyst comprising at least one group 10 metal impregnated in a mixed metal oxide, wherein the mixed metal oxide is a combination of In₂O₃, CuO, ZnO, ZrO₂, and Al₂O₃.

In an embodiment of the present disclosure, there is provided a catalyst as described herein, wherein the In₂O₃ has weight percentage in the range of 1-5% with respect to the total catalyst; CuO has a weight percentage in the range of 50-59% with respect to the total catalyst; ZnO has a weight percentage in the range of 10-20% with respect to the total catalyst; ZrO₂ has a weight percentage in the range of 5-10% with respect to the total catalyst; and Al₂O₃ has a weight percentage in the range of 5-10% with respect to the total catalyst. In another embodiment of the present disclosure, In₂O₃ has weight percentage in the range of 2-4% with respect to the total catalyst; CuO has a weight percentage in the range of 55-59% with respect to the total catalyst; ZnO has a weight percentage in the range of 15-20% with respect to the total catalyst; ZrO₂ has a weight percentage in the range of 8-10% with respect to the total catalyst; and Al₂O₃ has a weight percentage in the range of 8-10% with respect to the total catalyst.

In an embodiment of the present disclosure, there is provided a catalyst comprising at least one group 10 metal impregnated in a mixed metal oxide, wherein the mixed metal oxide is a combination of In₂O₃, CuO, ZnO, ZrO₂ and Al₂O₃, and wherein In₂O₃ has weight percentage in the range of 1-5% with respect to the total catalyst; CuO has a weight percentage in the range of 50-59% with respect to the total catalyst; ZnO has a weight percentage in the range of 10-20% with respect to the total catalyst; ZrO₂ has a weight percentage in the range of 5-10% with respect to the total catalyst; and Al₂O₃ has a weight percentage in the range of 5-10% with respect to the total catalyst.

In an embodiment of the present disclosure, there is provided a catalyst as described herein, wherein the group 10 metal is Pd, with a weight percentage in the range of 0.1-2% with respect to the total catalyst.

In an embodiment of the present disclosure, there is provided a catalyst comprising Pd impregnated in a mixed metal oxide selected from the group consisting of the oxides of In, Cu, Zn, Zr, Al, and combinations thereof, wherein Pd has a weight percentage in the range of 0.1-2% with respect to the total catalyst.

In an embodiment of the present disclosure, there is provided a catalyst comprising Pd impregnated in mixed metal oxide, wherein the mixed metal oxide is a combination of In₂O₃, CuO, ZnO, ZrO₂, and Al₂O₃.

In an embodiment of the present disclosure, there is provided a catalyst comprising Pd impregnated in a mixed metal oxide, wherein the mixed metal oxide is a combination of In₂O₃, CuO, ZnO, ZrO₂, and Al₂O₃, wherein Pd has a weight percentage in the range of 0.1-2% with respect to the total catalyst, and wherein the mixed metal oxide is a combination of In₂O₃, CuO, ZnO, ZrO₂, and Al₂O₃, and wherein In₂O₃ has weight percentage in the range of 1-5% with respect to the total catalyst; CuO has a weight percentage in the range of 50-59% with respect to the total catalyst; ZnO has a weight percentage in the range of 10-20% with respect to the total catalyst; ZrO₂ has a weight percentage in the range of 5-10% with respect to the total catalyst; and Al₂O₃ has a weight percentage in the range of 5-10% with respect to the total catalyst.

In an embodiment of the present disclosure, there is provided a catalyst as described herein, wherein the catalyst has a specific area in the range of 25-50 m²/g. In another embodiment of the present disclosure, the catalyst has a specific area in the range of 28-42 s m²/g.

In an embodiment of the present disclosure, there is provided a catalyst comprising Pd impregnated in a mixed metal oxide, wherein the mixed metal oxide is a combination of In₂O₃, CuO, ZnO, ZrO₂, and Al₂O₃, wherein the catalyst has a specific area in the range of 25-50 m²/g.

In an embodiment of the present disclosure, there is provided a process for preparing a catalyst as described herein, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture; d) processing the first reaction mixture to obtain a mixed metal oxide; e) contacting the mixed metal oxide and an aqueous solution of palladium nitrate to obtain a second solution; and f) processing the second solution to obtain the catalyst.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture; d) processing the first reaction mixture to obtain a mixed metal oxide; e) contacting the mixed metal oxide and an aqueous solution of palladium nitrate to obtain a third solution; and f) processing the third solution to obtain the catalyst.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture; d) processing the first reaction mixture to obtain a mixed metal oxide; e) contacting the mixed metal oxide and an aqueous solution of palladium nitrate to obtain a third solution; and f) processing the third solution to obtain the catalyst.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃ wherein In₂O₃ has weight percentage in the range of 1-5% with respect to the total catalyst; CuO has a weight percentage in the range of 50-59% with respect to the total catalyst; ZnO has a weight percentage in the range of 10-20% with respect to the total catalyst; ZrO₂ has a weight percentage in the range of 5-10% with respect to the total catalyst; and Al₂O₃ has a weight percentage in the range of 5-10% with respect to the total catalyst, and wherein the group 10 metal is Pd with weight percentage in the range of 0.1-2% with respect to the total catalyst, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture; d) processing the first reaction mixture to obtain a mixed metal oxide; e) contacting the mixed metal oxide and an aqueous solution of palladium nitrate to obtain a third solution; and f) processing the third solution to obtain the catalyst.

In an embodiment of the present disclosure, there is provided a process for preparing a catalyst as described herein, wherein the at least one precipitating agent is selected from the group consisting of sodium carbonate, potassium carbonate, ammonium carbonate and sodium hydrogen carbonate, and combinations thereof. In another embodiment, the at least one precipitating agent is sodium carbonate.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution, wherein the at least one precipitating agent is selected from the group consisting of sodium carbonate, potassium carbonate, ammonium carbonate and sodium hydrogen carbonate, and combinations thereof; c) contacting the first solution, and the second solution to obtain a first reaction mixture; d) processing the first reaction mixture to obtain a mixed metal oxide; e) contacting the mixed metal oxide and an aqueous solution of palladium nitrate to obtain a third solution; and f) processing the third solution to obtain the catalyst.

In an embodiment of the present disclosure, there is provided a process for preparing a catalyst as described herein, wherein contacting the first solution, and the second solution is carried out at a temperature in the range of 50-90° C. for a period in the range of 40-50 minutes to obtain a first reaction mixture. In another embodiment, contacting the first solution, and the second solution is carried out at a temperature in the range of 60-80° C. for a period in the range of 40-50 minutes to obtain a first reaction mixture.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution at a temperature in the range of 50-90° C. for a period in the range of 40-50 minutes to obtain a first reaction mixture to obtain a first reaction mixture; d) processing the first reaction mixture to obtain a mixed metal oxide; e) contacting the mixed metal oxide and an aqueous solution of palladium nitrate to obtain a third solution; and f) processing the third solution to obtain the catalyst.

In an embodiment of the present disclosure, there is provided a process for preparing a catalyst as described herein, wherein processing the first reaction mixture comprises processes selected from the group consisting of ageing, filtering, washing, drying, calcining, and combinations thereof to obtain the mixed metal oxide.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture to obtain a first reaction mixture; d) processing the first reaction mixture through processes selected from the group consisting of ageing, filtering, washing, drying, calcining, and combinations thereof, to obtain a mixed metal oxide; e) contacting the mixed metal oxide and an aqueous solution of palladium nitrate to obtain a third solution; and f) processing the third solution to obtain the catalyst.

In an embodiment of the present disclosure, there is provided a process for preparing a catalyst as described herein, wherein processing the third solution comprises processes selected from the group consisting of stirring, sonication, drying, calcination, extrusion, and combinations thereof.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture to obtain a first reaction mixture; d) processing the first reaction mixture, to obtain a mixed metal oxide; e) contacting the mixed metal oxide and an aqueous solution of palladium nitrate to obtain a third solution; and f) processing the third solution through processes selected from the group consisting of stirring, sonication, drying, calcination, extrusion, and combinations thereof to obtain the catalyst.

In an embodiment of the present disclosure, there is provided a process for preparing a catalyst as described herein, wherein processing the first reaction mixture comprises processes selected from the group consisting of ageing, filtering, washing, drying, calcining, and combinations thereof to obtain the mixed metal oxide, and wherein calcination is carried out at a temperature in the range of 300-450° C. at heating rates between 2-5° C./minute.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution to obtain a first reaction mixture to obtain a first reaction mixture; d) processing the first reaction mixture through processes selected from the group consisting of ageing, filtering, washing, drying, calcining, and combinations thereof, to obtain a mixed metal oxide, wherein calcination is carried out at a temperature in the range of 300-450° C. at heating rates between 2-5° C./minute; e) contacting the mixed metal oxide and an aqueous solution of palladium nitrate to obtain a third solution; and f) processing the third solution to obtain the catalyst.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution at a temperature in the range of 50-90° C. for a period in the range of 40-50 minutes to obtain a first reaction mixture to obtain a first reaction mixture; d) processing the first reaction mixture through processes selected from the group consisting of ageing, filtering, washing, drying, calcining, and combinations thereof, to obtain a mixed metal oxide, wherein calcination is carried out at a temperature in the range of 300-450° C. at heating rates between 2-5° C./minute; e) contacting the mixed metal oxide and an aqueous solution of palladium nitrate to obtain a third solution; and f) processing the third solution through processes selected from the group consisting of stirring, sonication, drying, calcination, extrusion, and combinations thereof to obtain the catalyst.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, wherein the mixed metal oxide is a combination of In₂O₃, CuO, ZnO, ZrO₂ and Al₂O₃, and wherein the group 10 metal is Pd, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution at a temperature in the range of 50-90° C. for a period in the range of 40-50 minutes to obtain a first reaction mixture to obtain a first reaction mixture; d) processing the first reaction mixture through processes selected from the group consisting of ageing, filtering, washing, drying, calcining, and combinations thereof, to obtain a mixed metal oxide, wherein calcination is carried out at a temperature in the range of 300-450° C. at heating rates between 2-5° C./minute; e) contacting the mixed metal oxide and an aqueous solution of palladium nitrate to obtain a third solution; and f) processing the third solution through processes selected from the group consisting of stirring, sonication, drying, calcination, extrusion, and combinations thereof to obtain the catalyst.

In an embodiment of the present disclosure, there is provided a process for preparing a catalyst comprising Pd impregnated in a mixed metal oxide, wherein the mixed metal oxide is a combination of In₂O₃, CuO, ZnO, ZrO₂, and Al₂O₃, the In₂O₃ has weight percentage in the range of 1-5% with respect to the total catalyst; CuO has a weight percentage in the range of 50-59% with respect to the total catalyst; ZnO has a weight percentage in the range of 10-20% with respect to the total catalyst; ZrO₂ has a weight percentage in the range of 5-10% with respect to the total catalyst; and Al₂O₃ has a weight percentage in the range of 5-10% with respect to the total catalyst, Pd with a weight percentage in the range of 0.1-2% with respect to the total catalyst, said process comprising: a) contacting at least one metallic nitrate selected from the group consisting of indium nitrate, copper nitrate, zinc nitrate, zirconium nitrate, aluminum nitrate, and combinations thereof, and deionized water to obtain first solution; b) contacting at least one precipitating agent, and deionized water to obtain a second solution; c) contacting the first solution, and the second solution at a temperature in the range of 50-90° C. for a period in the range of 40-50 minutes to obtain a first reaction mixture to obtain a first reaction mixture; d) processing the first reaction mixture through processes selected from the group consisting of ageing, filtering, washing, drying, calcining, and combinations thereof, to obtain a mixed metal oxide, wherein calcination is carried out at a temperature in the range of 300-450° C. at heating rates between 2-5° C./minute; e) contacting the mixed metal oxide and an aqueous solution of palladium nitrate to obtain a third solution; and f) processing the third solution through processes selected from the group consisting of stirring, sonication, drying, calcination, extrusion, and combinations thereof to obtain the catalyst.

The catalyst after synthesis was further extruded prior to loading the catalyst onto a reactor where both reduction and activation was coupled in a single step, to provide for improved catalytic activity, energy efficiency, and reduced time. Extrusion of the catalyst was found to be a very important step where, the nature, percentage of the binder, extrusion size was found to be crucial parameters for the extrudate strength as well as the activity and stability of the catalyst. Pseudoboehmite (PSBH) possessing cooperative catalytic effect has been identified as the best binder to impart long run stability to the catalyst. PSBH is an aluminum-based compound having composition AlO(OH). It consists of finely crystalline boehmite having higher water content. PSBH consists of similar octahedral layers in the xz plane as in boehmite lacking 3-D ordering due to restricted number of unit cells along y direction. [Tettenhorst, R., Crystal Chemistry of Boehmite, Clays and Clay Minerals, Vol. 28, No. 5, 373-380, (1980)]. On heating, PSBH transforms to γ-alumina with unchanged pore size disposition up to 1000° C. [Nogi, K., Nanoparticle Technology Handbook, S. 204]. In an embodiment of the present disclosure, the catalyst comprises a binder selected from a group consisting of PSBH, organic binders, plasticizers, silica sols, alumina, HZSM's, polymeric binders, clays, and aluminum phosphate. In another embodiment of the present disclosure, the binder is PSBH, and wherein, the binder has a weight percentage in the range of 20-40% of the catalyst. In another embodiment, the weight percentage of the binder is in the range of 27-32% of the catalyst.

In an embodiment of the present disclosure, the catalyst further comprises a co-binder selected from a group consisting of concentrated nitric acid (HNO₃), acetic acid, phosphoric acid, distilled water, polyvinyl alcohol, wherein the co-binder has a weight percentage in the range of 4-8% of the binder. In another embodiment of the present disclosure, the co-binder is concentrated nitric acid having a weight percentage in the range of 5-7% of the binder.

The catalyst thus extruded is further used to for thermochemical conversion of CO₂, where the thermochemical conversion of CO₂ to methanol (MeOH) and carbon monoxide (CO) is performed inside a reactor. For this purpose, the gases (CO₂ and H₂) were passed from cylinders into a reactor having fixed catalyst bed. Before reaching the fixed catalyst bed, the gases are pre-heated to 200° C. CO₂ and H₂ were used in defined molar ratios in a continuous down flow vapor phase reactor under appropriate reaction conditions, to maximize the catalytic conversion and selectivity for the desired product (MeOH and CO₂). In an embodiment of the present disclosure, there is provided a process for thermochemical CO₂ reduction, the process comprising: a) activating the catalyst comprising at least one group 10 metal impregnated in a mixed metal oxide selected from the group consisting of the oxides of In, Cu. Zn, Zr, Al, and combinations thereof in a hydrogen stream to obtain an activated catalyst; and b) contacting H₂, CO₂ and the activated catalyst at a reaction temperature in the range of 200° C. to 300° C. under a reaction pressure of 30-60 bar with a space velocity of 1000-7200 h⁻¹ to thermochemically reduce CO₂. In another embodiment of the present disclosure, there is provided a process for thermochemical CO₂ reduction, the process comprising: a) activating the catalyst comprising at least one group 10 metal impregnated in a mixed metal oxide selected from the group consisting of the oxides of In, Cu. Zn, Zr, Al, and combinations thereof in a hydrogen stream to obtain an activated catalyst; and b) contacting H₂, CO₂ and the activated catalyst at a reaction temperature in the range of 250° C. to 300° C. under a reaction pressure of 40-50 bar with a space velocity of 3000-5000 h⁻¹ to thermochemically reduce CO₂.

In an embodiment of the present disclosure, there is provided use of the catalyst as described herein, for thermochemical CO₂ reduction.

In an embodiment of the present disclosure, there is provided a catalyst as as described for use in thermochemical CO₂ reduction.

In an embodiment of the present disclosure, there is provided a process for thermochemical CO₂ reduction with a CO₂ conversion efficiency in the range of 25-38%.

In an embodiment of the present disclosure, there is provided a process for thermochemical CO₂ reduction, the process comprising: a) activating the catalyst comprising at least one group 10 metal impregnated in a mixed metal oxide selected from the group consisting of the oxides of In, Cu. Zn, Zr, Al, and combinations thereof in a hydrogen stream to obtain an activated catalyst; and b) contacting H₂, CO₂ and the activated catalyst at a reaction temperature in the range of 200° C. to 300° C. under a reaction pressure of 30-60 bar with a space velocity of 1000-7200 h⁻¹ to thermochemically reduce CO₂, with a CO₂ conversion efficiency in the range of 25-38%.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

The working examples described herein clearly depict a convenient process for the synthesis of catalyst, which allows efficient thermochemical conversion of CO₂ to methanol and carbon monoxide. The conversion efficiency and selectivity demonstrated by the catalysts of the present disclosure allow for a greater yield of value-added products like methanol and carbon monoxide, with fewer by-products. Accordingly, the present disclosure described a catalyst comprising at least one group 10 metal impregnated in a mixed metal oxide selected from the group consisting of the oxides of In, Cu, Zn, Zr, Al, and combinations thereof

### Materials and Methods

Cu(NO₃)₂.6H₂O, Zn(NO₃)₂.2H₂O were procured from Alfa Aesar; Na₂CO₃ was procured from SDFCL; In(NO₃)₃ and Pd(NO₃)₂ were procured from Sigma Aldrich; UHP CO₂ (99.999), UHP H₂ (99.999); UHP N₂ (9.99); calibration gas cylinders were procured from Chemix; and isopropyl alcohol was procured from Merck. All the chemicals were obtained from high grade certified reagent houses and were used without further purification.

The catalyst of the present disclosure, as synthesized in Example 1 and Example 2, was characterized by Powder X-ray Diffraction (PXRD), Energy Dispersive X-ray Analysis (EDAX), TEM and high resolution TEM (HRTEM), Selected Area Electron Diffraction (SAED), and gas and liquid chromatography. Powder X-ray Diffraction (PXRD) measurements were done at room temperature on a Rigaku Miniflex X-ray diffractometer with Cu-Kα X-ray source (λ=1.5406 Å), equipped with a position sensitive detector in the angular range 20°<20<80° with the step size 0.02° and scan rate of 0.5 s/step calibrated against corundum standards. Quantitative microanalysis on all the samples were performed with a FEI NOVA NANOSEM 600 instrument equipped with an EDAX® instrument. Data were acquired with an accelerating voltage of 20 kV and a 100 s accumulation time.

The Energy Dispersive X-ray Analysis (EDAX) analysis was performed using P/B-ZAF standard less method (where, Z=atomic no. correction factor, A=absorption correction factor, F=fluorescence factor, PB=peak to background model) on selected spots and points. TEM and high resolution TEM (HRTEM), selected area electron diffraction (SAED) patterns were collected using a JEOL 200 TEM instrument. Samples for these measurements were prepared by dropping a small volume of sonicated ethanolic dispersion onto a carbon-coated copper grid.

All the gas and liquid analyses were done using an online Agilent gas chromatography system (7890B). Gas analyses were done online using a Thermal

Conductivity Detector (TCD) detector and a Heysep and Mol Sieve column. The liquid sample analyses with internal standard were done using an automatic liquid injector through a capillary column and Flame Ionization Detector (FID) detector. The temperature programmed reduction (TPR) studies were done on a TPD/TPR machine equipped with a TCD detector. Ultra-high purity (UHP) hydrogen was passed over a fixed amount catalyst the stream of which was recorded on a TCD detector between 30 and 800° C.

Calibration: The calibration of CO₂, CO, H₂ and N₂ were done by using standard gases and calibrated Brooks MFCs for corresponding gases. The individual gas percentages were determined from gas chromatograms peak areas using N₂ as internal standard. The MeOH in the liquid products was calibrated by using isopropyl alcohol as internal standard. All the reactor parts used for calibration and analyses were accurately calibrated before use, such as MFC from Brooks, wet gas meter from Ritter, weigh scale from Mettler Toledo.

### Example 1

### Synthesis of the Catalyst

167 g of Cu(NO₃)₂ salt was dissolved in 37.5 mL deionized water; 109 g of Zn(NO₃)₂ salt was dissolved in 37.5 mL deionized water; 5.4 g of indium nitrate dissolved in 37.5 mL deionized water; 9.4 g of Zr(ONO₃)₂ dissolved in 37.5 mL deionized water and 18.4 g Al(NO₃)₃ was dissolved in 37.5 mL deionized water; were all mixed to obtain a first solution. Further, at least one precipitating agent, 1M of Na₂CO₃ (7.9 g), was dissolved in 75 mL of deionized water to obtain a second solution. Furthermore, the first solution and the second solution were mixed dropwise in a three necked-flasks with 150 mL deionized water, at a temperature of 70° C. in an oil bath, through constant stirring, for a period of 40-50 minutes to obtain a first reaction mixture. The pH of the first reaction mixture was maintained in the range of 7-8 to smoothen the precipitation process. The first reaction mixture was further allowed to be heated to a temperature of 70° C. for 40-50 minutes, without stirring. The reaction flask containing the first reaction mixture (precipitate) was then aged for 60-90 minutes above the oil bath. The first reaction mixture (precipitate) was further filtered, washed with distilled water to remove the unwanted metal ions. The precipitate was dried in a vacuum oven at 90° C. temperature for overnight. The sample was further calcined at 350° C. temperature for 3 hours to obtain a mixed metal oxide, i.e., In₂O₃, CuO, ZnO, ZrO₂ and Al₂O₃. The mixed metal oxide was suddenly quenched by bringing the mixed metal oxide to room temperature to provide thermal shocks to create defects and nano-structuring. This nano-structuring reduced the cluster size of the catalytically active Cu centers and increases its effective surface area. The mixed metal oxide was further impregnated with an aqueous solution Pd(NO₃)₂ salt to impregnate palladium particles onto the mixed metal oxide to obtain a third solution. The aqueous solution Pd(NO₃)₂ salt was prepared by dissolving 2.2 g of Pd(NO₃)₂ salt in 95 ml of deionized water (0.1M). The catalyst (100 g) was prepared by alternate cycles of vigorous stirring and sonication of the mixed metal oxide for 2 hours followed by drying under continuous stirring. The impregnated mixture was vacuum dried for 12 hours and calcined at temperatures between 300-450° C., at heating rates of 2-5° C./min, under a hydrogen flow of 10 to 50% hydrogen content, to obtain 100 g of the catalyst.

### Example 2

### Extrusion of the Catalyst

The catalyst as prepared in the Example 1 was further extruded with a binder, pseudoboehmite (PSBH), in 20-40 wt. % of the catalysts; and a co-binder, i.e., conc. HNO₃, in the range of 2-10 wt % with respect to the binder. For this purpose, the catalyst was mixed using a mortar pestle with the PSBH, and a slurry was obtained by dropwise addition of the HNO₃ and H₂O. The slurry was extruded in a Na wire press or extrusion machine to yield extrudates of length between 0.5-2 cm and diameter of 1-2 mm. These extrudates were dried at a temperature range of 25-37° C. and subsequently baked at 100-150° C. for 12-15 hours before reduction of the catalyst inside a reactor.

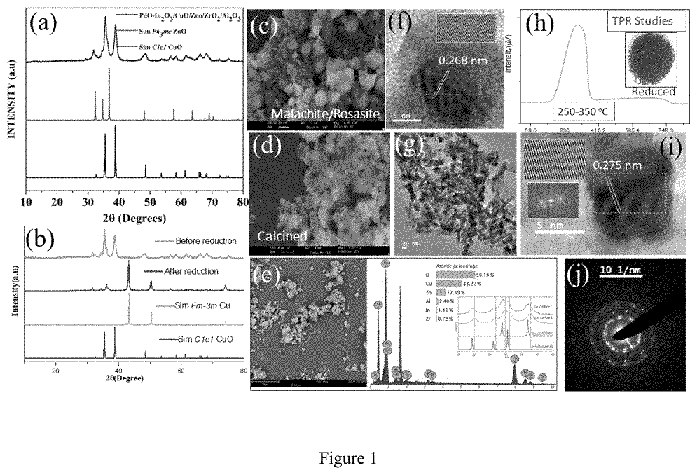

FIG. 1(a) illustrates characterization data of the catalyst (a) Powder X-ray Diffraction (PXRD) of PdO—In₂O₃/CuO/ZnO/ZrO₂/Al₂O₃ (catalyst) and comparison with bulk oxides. Comparison of the experimental PXRD with the simulated pattern of the metal oxides clearly shows the formation of the phases without any impurity. A combination of the individual peaks of CuO and ZnO phases in the catalyst PXRD is consistent with the formation of the two major phases. The compositional percentages of Pd/In₂O₃ are too low to be reflected in the diffraction patters. FIG. 1(b) illustrates characterization by PXRD after reduction of the catalyst. Shifts in the peak positions of the CuO phase indicates secondary metal doping in the CuO lattice as well as the presence of oxygen vacancies. Doping of the secondary metals and introduction of vacancies enhances the catalytic activity improving the reducibility of the catalyst and adsorption of the reaction molecules. The creation of oxide vacancies is enhanced by sudden thermal shock by quenching the catalyst at the annealing step from high temperature (350-450° C.) directly to room temperature.

The catalyst, before and after calcination, was characterized by Scanning Electron Microscopy (SEM), as can be observed from FIGS. 1(c) and 1(d), respectively. The samples before and after calcination were imaged to understand the microstructure of the phases. Changes in the morphology upon calcination shows the effect of calcination in forming a microstructure mixed oxide catalyst. FIG. 1(e) depicts SEM and Energy-Dispersive X-ray Spectroscopy (EDS) of the mixed metal oxide, i.e., In₂O₃, CuO, ZnO, ZrO₂ and Al₂O₃. EDS was used in conjunction with SEM to for elemental analysis/chemical characterization of the sample. The atomic percentage of each atom in the catalyst can be observed from the FIG. 1e. The percentage of the oxygen in the catalyst was found to be 50.16%, Cu was 33.22%, Zn was 12.39%, Al was 2.4%, In was 1.11%, and Zr was 0.72%.

FIG. 1(f) depicts High-Resolution Transmission Electron Microscopy (HRTEM) images of the catalyst. Transmission electron microscopy (TEM) was used to image the catalyst in the nano-regime where presence of metal particles was identified on connected metal oxide supports, as can be observed in FIG. 1(g). Temperature programmed reduction profile of the catalyst using H₂ as the reductant shows the reduction temperature of the catalyst which is between 250 and 300° C., as can be observed in FIG. 1(h). The HRTEM and SAED patterns indicates the crystallinity of the catalyst and shows the characteristic d-spacing of the crystalline phases (110 plane of CuO), as can be observed in FIGS. 1(i) and 1(j). It was observed that the interplanar distance (d-spacing of the lattice fringes) of the resolved particle corresponding to the CuO phase is 0.268 nm.

### Example 3

### Thermochemical CO₂ Conversion

The synthesized catalyst was used for thermo-catalytic conversion of CO₂ to methanol (MeOH) and carbon monoxide (CO). For this purpose, the gases (CO₂ and H₂) were passed from cylinders into a reactor having fixed catalyst bed. Before reaching the fixed catalyst bed, the gases are pre-heated to 200° C. CO₂ and H₂ were used in a molar ratio of 1:3 with GHSV=1000-3600 h⁻¹ in a continuous down flow vapor phase reactor at an operating pressure in the range of 30-50 bar′ and an operating temperature of 250-300° C., to maximize the catalytic conversion and selectivity for the desired product (MeOH and CO₂). Water was formed as a by-product of this reaction. The catalyst loaded inside the reactor was activated at a temperature of about 350° C. for 3-4 hours using low concentration of H₂ flow. The catalyst was cooled down to room temperature further to which the reaction was started. During the reaction the constant flow of reactant gases, pressure and temperature of the system were maintained using calibrated Brooks MFCs, pressure indicators, internal temperature sensor, all of which were integrated over a SCADA interface. The increase of temperature beyond the set value was the result of the exothermicity of the reaction.

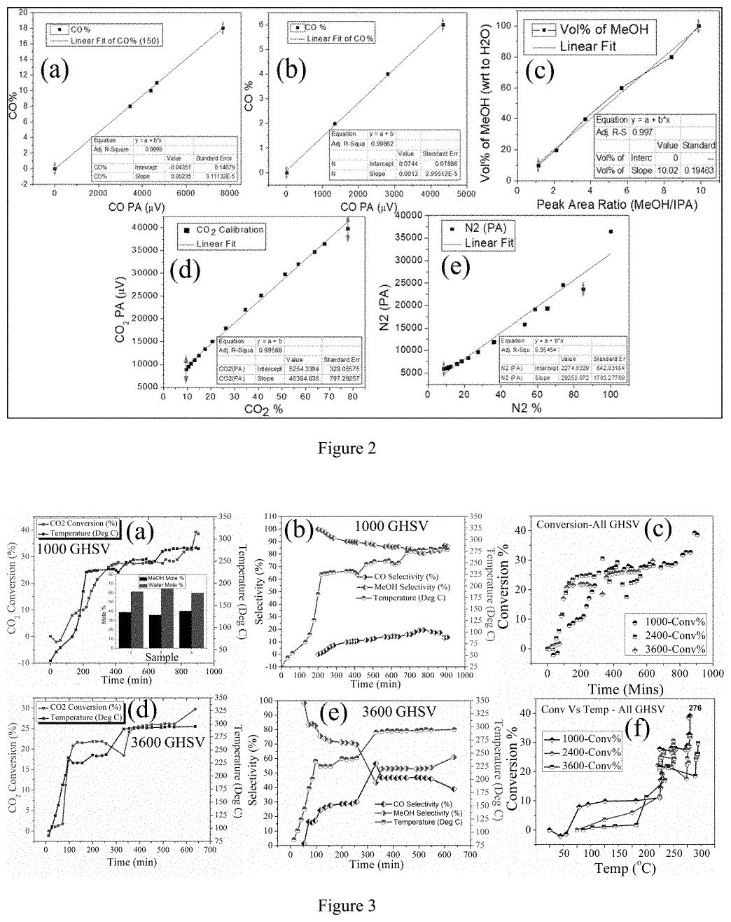

The liquid products (MeOH and H₂O) were separated using heat exchanger and phase separator modules; and were collected at regular time intervals and analysed using a Poraplot Q capillary column and FID detector. For analysis, 100 μL of IPA was added to 1 ml aliquot of the condensed liquid products (MeOH and H₂O) for GC analysis. Calibration curve for liquid mixture analysis was obtained by using 5 concentration values of MeOH ranging between 0-100% using 1 ml of solution and 100 μL IPA as the internal standard. Similarly, the converted gaseous products (CO, unreacted CO₂ and H₂) were analysed through gas chromatography. Nitrogen (2%) was used as the internal standard for the gaseous analysis and the correction of product and input gases. The online gas analysis was done using a Haysep and molecular sieve columns and TCD detector. The various stages of the catalyst design are depicted in FIG. 2, i.e., calibration curves for (a) CO (concentration range: 0-20%), (b) CO (concentration range: 0-10%), (c) MeOH (using IPA as the internal standard), (d) CO₂ and (e) N₂. All calibrations were done using certified calibration mixtures. All the graphs in FIG. 2 correspond to the calibrations of the different gases at different concentration ranges in the gas chromatography (GC) system. These are plots of GC peak area vs. concentration of a gas. Thus, all these calibration curves were used to determine the unknown concentration of gases in a mixture by analysing the corresponding GC peak area. The different components of product analysis are herewith provided:

CO₂ Conversion Calculation:

$${CO}_{2} conversion ( \% ) = 1 - ( \frac{{CO}_{2 output}}{{CO}_{2 input}} ) ( \frac{{N}_{2 input}}{{N}_{2 output}} ) * 100 \qquad Eq ( 3 )$$

CO₂ (input) or N₂ (input) has been taken as CO₂ and N₂ values at T₀. The T₀ values were taken as the GC reading just before CO formation started. CO₂ out is taken as the value of CO₂ peak area at any given instant at the post-equilibrium state. CO₂ conversion was calculated after doing N₂ correction of the CO₂ peak area at time Tt.

CO (%) Calculation

The % of CO was calculated based on the corresponding peak area of CO in GC analyses and calibration curve supplied in the calibration section of the data Equation 4.

CO %=(CO peak area)*0.00138 (Slope)+0.06 (Intercept) Eq (4)

The average CO concentration varied between 1-2.5%.

Product (%) Selectivity Calculation

The selectivity's for the CO and MeOH were calculated by the equations given below. CO selectivity was calculated from the ratio of CO % in the product stream to the total calculated product formation at that point of time.

$$\begin{matrix} Output Flow ( O . F ) = Input Flow ( I . F ) * ( \frac{{N}_{2 input}}{{N}_{2 output}} ) & Eq ( 5 ) \\ CO selectivity ( \% ) = ( \frac{Output flow * CO \%}{[ I . F * {CO}_{2 input} ] - [ O . F * {CO}_{2 output} ]} ) * 100 & Eq ( 6 ) \\ CO \% = ( CO peak area ) * 0.00138 ( Slope ) + 0.06 ( Intercept ) & Eq ( 7 ) \\ {CO}_{2} \% = ( \frac{( {CO}_{2} peak area ) * ( \frac{{N}_{2 output}}{{N}_{2 input}} ) - 5254 ( intercept )}{46394 ( slope )} ) * 100 & Eq ( 8 ) \\ Methanol selectivity = 100 - CO selectivity \% & Eq ( 9 )\end{matrix}$$

The percentage conversion and selectivity vs. time and temperature at different Gas Hourly Space Velocity (GHSV) 72-hour steady state was plotted, and the results are presented in FIG. 3. The average conversion based on feed gas calculation in the 1000 GHSV reaction between 250-275° C. was found to be ˜28-38%, as can be observed in FIG. 3(a), while that in 3600 GHSV was around 22-25%, as can be observed from FIGS. 3(c), 3(d), and 3(f). Further, the average CO selectivity of the catalyst was found to be about 11-20% at 1000 GHSV, as can be observed in FIG. 3(b), whereas the CO selectivity was found to be much higher, in the range of 10-55% at 3600 GHSV, as can be observed in FIG. 3(e). The methanol selectivity was found to be in the range of 50-90%, at 1000, 2400, and 3600 GHSV (as can be observed in FIGS. 3(b) and 3(e).

### Example 4

### Liquid Analysis and MeOH Mole Percent Calculation

MeOH mole percent in the liquid product was calculated from the calibration curve (supplied) using IPA as the internal standard. MeOH/IPA peak area was calibrated against the volume percentage of MeOH. The average mole percent of MeOH was about 38%, as can be observed below in Table 1.

TABLE 1

| | MeOH | | MeOH/ | | | | | Mole | |
|---|---|---|---|---|---|---|---|---|---|
| | Peak | IPA | IPA | Vol % | Vol % | Moles | Moles | % of | Mole |
| | Area | Area | Area | MeOH | H₂O | of H₂O | of MeOH | MeOH | % H₂O |
| Sample 1 | 4825.2 | 821.6 | 5.87 | 58.85 | 41.15 | 50.984 | 32.479 | 38.91 | 61.085 |
| Sample 2 | 4239.7 | 763.2 | 5.55 | 55.66 | 44.34 | 57.885 | 32.375 | 35.868 | 64.131 |
| Sample 3 | 3521 | 585.7 | 6.01 | 60.24 | 39.76 | 47.716 | 32.202 | 40.294 | 59.706 |

Each of the parameters as listed in listed in Table 1 were calculated using the following formula, as mentioned in the equations 10-17.

$$\begin{matrix} MeOH Vol ( {V}_{MeOH} ) ( L ) = {V}_{MeOH} \% * {V}_{Total} & Eq ( 10 ) \\ Wt . of Methanol ( {W}_{MeOH} ) ( Kg ) = Density ( 0.792 Kg \text{/} L ) * {V}_{MeOH} & Eq ( 11 ) \\ Number of moles of methanol ( {n}_{MeOH} ) = ( \frac{{W}_{MeOH}}{M . {W}_{MeOH}} ) * 1000 & Eq ( 12 ) \\ {V}_{H 2 O} ( L ) = {V}_{H 2 O} \% * {V}_{total} & Eq ( 13 ) \\ Wt . of water ( {W}_{water} ) ( Kg ) = Density ( 1 Kg \text{/} L ) * {V}_{H 2 O} & Eq ( 14 ) \\ Number of moles of water ( {n}_{H 2 O} ) = ( \frac{{W}_{H 2 O}}{M . {W}_{H 2 O}} ) * 1000 & Eq ( 15 ) \\ Mole percentage ( MeOH ) = ( \frac{1}{1 + ( \frac{n . H 2 O}{n . MeOH} )} ) * 100 & Eq ( 16 ) \\ Mole percentage ( water ) = ( \frac{1}{1 + ( \frac{n . MeOH}{n . H 2 O} )} ) * 100 & Eq ( 17 )\end{matrix}$$

### Example 5

### Comparative Data for Catalyst Activity

The catalyst of the present disclosure was compared to the conventionally used catalysts to evaluate the performance of the catalyst for CO₂ conversion (%), CO selectivity (%), and MeOH selectivity (%). A comparative catalyst table is herewith provided below in Table 2.

TABLE 2

| | CO₂ | CO | MeOH | |
|---|---|---|---|---|
| | conversion | selectivity | selectivity | |
| Catalyst | (%) | (%) | (%) | Reference |
| Cu/ZnO/ZrO₂ | 23 | 43.2 | 56.8 | Applied Catalysis B: |
| | | | | Environmental 191 |
| | | | | (2016) 8-17 |
| Cu/Mo₂C | 28 | 35 | 26 | Catal. Sci. Technol., |
| | | | | 2016, 6, 6766-6777 |
| Cu/ZnO/Al₂O₃ | 9 | | 100 | Journal of CO₂ |
| | | | | Utilization 15 (2016) 83-88 |
| Pd/ZnO | 10.7 | 39 | 60 | Journal of Catalysis 343 |
| | | | | (2016) 133-146 |
| Cu/ZnO/Al₂O₃ | 10.1 | 78.2 | 21.8 | Reac Kinet Mech Cat |
| | | | | (2013) 110:131-145 |
| Au/CuO/SBA-15 | 24 | | 14 | J Porous Mater (2017) |
| | | | | 591-599 |
| Cu/MgO—TiO₂ | 5 | | 40 | Journal of Molecular |
| | | | | Catalysis A: Chemical |
| | | | | 425 (2016) 86-93 |
| CuO/ZnO/Al₂O₃ | 16.2 | | 63.8 | Fuel 164 (2016) 191-198 |
| In₂O₃ | 7.1 | | 39.7 | Journal of CO2 |
| | | | | Utilization 12 (2015) 1-6 |
| Cu/ZrO₂/CNF | 14 | | 75 | Catalysis Today 259 |
| | | | | (2016) 303-311 |
| CuO—ZnO—ZrO₂ | 24 | | 48 | Rare Met. (2016) |
| | | | | 35(10):790-796 |
| Pd—ZnO/CNT | 5.5 | | 99.8 | Catal Lett (2015) |
| | | | | 145:1138-1147 |
| Au/Cu—Zn—Al | 28 | | 60 | Applied Catalysis A: |
| | | | | General 504 (2015) 308- |
| | | | | 318 |
| Ga/Pd/β—Ga₂O₃ | <1 | | 52 | J. Catal., 292 (2012), pp. |
| | | | | 90-98 |
| Pd/ZnO | 11.4 | | 50 | Appl. Catal. A Gen., 125 |
| | | | | (1995), pp. L199-L202 |
| Cu/Zn/Al/ZrO₂ | 18.7 | 52.76 | 47.2 | Catal. Lett., 118 (2007), |
| | | | | pp. 264-269 |
| PdO- | 28-38 | 11-20 | 80 | Present disclosure |
| In₂O₃/CuO/ZnO/ | | | | |
| ZrO₂/Al₂O₃ | | | | |

Based on the data provided in Table 2, it can be inferred that CO₂ conversion through the catalyst of the present disclosure was found to be in the range of 25-38%, which is much higher than the catalysts of the prior art. It may be noted that the inclusion of zirconium oxide provided improved stability to the catalyst of the present disclosure. Further, the methanol selectivity and the CO selectivity was found to be much higher than the conventionally used catalysts. Also, the catalysts of the prior art, which exhibited good selectivity towards methanol formation, have shown very poor selectivity towards the formation of carbon monoxide. However, unlike the conventionally used catalysts, the catalysts of the present disclosure showed a good percentage selectivity towards formation of methanol and carbon monoxide, and the same has been experimentally established as described herein.

### Advantages of the Present Disclosure:

The present disclosure discloses a catalyst comprising at least one group 10 metal impregnated in a mixed metal oxide selected from the group consisting of the oxides of In, Cu. Zn, Zr, Al, and combinations thereof. The catalysts of the present disclosure allow for efficient thermo-chemical conversion of CO₂ to value added products like methanol and carbon monoxide with enhanced conversion efficiency and selectivity. The selectivity of the catalyst towards methanol formation was around 80%, which is much higher in comparison to the conventionally used catalysts such as Cu/ZnO/ZrO₂, Pd/ZnO, Cu/ZnO/Al₂O₃, Pd/CNT's, etc. Also, the percentage CO₂ conversion through the use of catalysts of the present disclosure is in the range of 20-40% which is much higher in comparison to the conventionally used catalysts such as Cu/ZnO/Al₂O₃, Pd/ZnO, Cu/ZnO, Pd/ZnO/Al₂O₃, In₂O₃, to name a few. Also, the catalyst of the present disclosure shows improved stability due to the inclusion of ZrO₂. The catalyst of the present disclosure is obtained by a convenient process, which does not require the use of extreme operating conditions. The present disclosure provides for a catalyst comprising at least one group 10 metal impregnated in a mixed metal oxide selected from the group consisting of the oxides of In, Cu. Zn, Zr, Al, and combinations thereof. The catalyst of the present disclosure is effective in conversion of CO₂ to value added products like methanol (CH₃OH) and carbon monoxide (CO). The present disclosure further discloses a convenient process for preparation of the catalyst.

It will be appreciated by those skilled in the art that changes could be made to the embodiment described above without departing from the broad inventive concept thereof. It is understood, therefore, that this invention is not limited to the particular embodiments disclosed, but it is intended to cover modifications within the spirit and scope of the present invention as defined by the appended claims.

1-15. (canceled)

16. A catalyst comprising at least one group 10 metal impregnated in a mixed metal oxide, wherein the mixed metal oxide is a combination of In₂O₃, CuO, ZnO, ZrO₂, and Al₂O₃.

17. The catalyst as claimed in claim 16, wherein the In₂O₃ has weight percentage in the range of 1-5% with respect to the total catalyst; CuO has a weight percentage in the range of 50-59% with respect to the total catalyst; ZnO has a weight percentage in the range of 10-20% with respect to the total catalyst; ZrO₂ has a weight percentage in the range of 5-10% with respect to the total catalyst; and Al₂O₃ has a weight percentage in the range of 5-10% with respect to the total catalyst.

18. The catalyst as claimed in claim 16, wherein the at least one group 10 metal is Pd having a weight percentage in the range of 0.1-2% with respect to the catalyst.

19. The catalyst as claimed in claim 16 has a specific area in the range of 25-50 m²/g.

20. A process of preparation of catalyst as claimed in claim 16, the process comprising:
(a) contacting at least one metallic nitrate selected from the group consisting of indium nitrate, copper nitrate, zinc nitrate, zirconium nitrate, aluminum nitrate, and combinations thereof, and deionized water to obtain first solution;
(b) contacting at least one precipitating agent, and deionized water to obtain a second solution;
(c) contacting the first solution, and the second solution to obtain a first reaction mixture;
(d) processing the first reaction mixture to obtain a mixed metal oxide;
(e) contacting the mixed metal oxide and an aqueous solution of palladium nitrate to obtain a third solution;
(f) processing the third solution to obtain the catalyst.

21. The process as claimed in claim 20, wherein the at least one precipitating agent is selected from the group consisting of sodium carbonate, potassium carbonate, ammonium carbonate and sodium hydrogen carbonate, and combinations thereof.

22. The process as claimed in claim 20, wherein contacting the first solution, and the second solution is carried out at a temperature in the range of 50-90° C. for a period in the range of 40-50 minutes to obtain a first reaction mixture.

23. The process as claimed in claim 20, wherein processing the first reaction mixture comprises processes selected from the group consisting of ageing, filtering, washing, drying, calcining, and combinations thereof to obtain the mixed metal oxide.

24. The process as claimed in claim 20, wherein processing the third solution comprises processes selected from the group consisting of stirring, sonication, drying, calcination, extrusion, and combinations thereof.

25. The process as claimed in claim 23, wherein calcination is carried out at a temperature in the range of 300-450° C. at heating rates between 2-5° C./minute.

26. A process for thermochemical CO₂ reduction, the process comprising using the catalyst as claimed in claim 16.

27. A catalyst as claimed in claim 16, for use in thermochemical CO₂ reduction.

28. A process for thermochemical CO₂ reduction, the process comprising:
(a) activating the catalyst as claimed in claim 16 in a hydrogen stream to obtain an activated catalyst; and
(b) contacting H₂, CO₂ and the activated catalyst at a reaction temperature in the range of 200° C. to 300° C. under a reaction pressure of 30-60 bar with a space velocity of 1000-7200 h-1 to thermochemically reduce CO₂.

29. The process as claimed in claim 28, provides a conversion efficiency of CO₂ in the range of 25-38%.
